(19) United States
(12) Patent Application Publication
(10) Pub. No.: US 2010/0063596 A1
(43) Pub. Date: Mar. 11, 2010

(54) JOINT SOCKET AND HIP ENDOPROSTHESIS HAVING THE SAME

(75) Inventor: Martin Imhof, Meggen (CH)

Correspondence Address: KNOBBE MARTENS OLSON & BEAR LLP, 2040 MAIN STREET, FOURTEENTH FLOOR, IRVINE, CA, 92614, US

(73) Assignee: Smith & Nephew Orthopaedics AG, Rotkreuz (CH)

(21) Appl. No.: 12/296,796

(22) PCT Filed: Mar. 6, 2007
(86) PCT No.: PCT/EP2007/001919

§ 371 Date: May 18, 2009

(30) Foreign Application Priority Data
Apr. 13, 2006 (DE) 10 2006 017 473.9

(51) Int. Cl. A61F 2/34

(52) U.S. Cl. 623/22.22

(57) ABSTRACT

Joint socket, especially for a hip endoprosthesis, having a socket shell (18) and a socket insert (20) for mounting a joint head. The socket insert (20) has a portion (22) having a spherical outer face (28) and is positionable in a receiving space (24) of the socket shell (18) in such a way that the spherical outer face (28) of the socket insert (20) is in contact with the inner face (30) of the receiving space (24) concentrically with the rotational axis thereof. In the region of concentric contact, the radius of curvature of the inner face (30) of the receiving space (24) of the socket shell (18) is always greater than the radius of curvature of the spherical portion (22) of the socket insert (20). As a result, self-locking clamping between socket insert and socket shell is achieved.

The resistance to deformation of at least one of the mutually corresponding faces (28, 30) is reduced with respect to that of the core region of the associated components, especially as a result of the formation of a surface structure (32, 34).

The invention relates to a joint socket, especially for a hip endoprosthesis, having a socket shell and having a socket insert for mounting a joint head, in accordance with the preamble of claim 1.

In total endoprostheses, especially hip total endoprostheses, a prosthesis stem having a joint head is inserted into the femur. A joint socket is implanted in the pelvic bone and serves as a bearing shell for the joint head. The joint socket usually comprises a socket shell and a socket insert. The socket shell is optimised in respect of the bone, while the socket insert governs the bearing properties of the joint head. The socket shell is shaped and positioned in the bone so as to allow the stablest possible incorporation of the socket shell into the bone. The socket insert can be so aligned in the socket shell that the joint head is received so that it gives as correct as possible an orthopaedic position of the prosthesis stem and thus of the femur of the patient.

EP 0 663 193 A1 discloses a joint socket in which the socket insert has a spherical outer face and is seated by means of that spherical outer face in a spherical receiving space having the same sphere radius of the socket shell. When the socket insert is inserted into the socket shell, the socket insert can be rotated as desired about its rotational axis and tilted with its rotational axis as desired with respect to the rotational axis of the receiving space. It is thus possible for the socket shell to be positioned in the bone in accordance with the bone structure. The socket insert can be aligned in accordance with the orthopaedic position of the prosthesis stem and joint head. In order to fixate the socket insert in its position in the socket shell, the spherical inner face of the receiving space of the socket shell has sharply projecting teeth which engage in the outer face of the socket insert. Because the teeth have to penetrate the outer face of the socket insert there are limitations in respect of the choice of material for the socket insert. The pressing of the socket insert onto the teeth of the socket shell makes it difficult to insert the socket insert in an exact position.

In order to avoid the above-mentioned problems, DE 103 60 390 A1 proposes that the outer face of the socket insert be in contact with the inner face of the receiving space along a line of contact concentric with the rotational axis of the receiving space; that the inner face of the receiving space narrow towards the pole of the receiving space in the region of that contact line in such a way that the radius of curvature in that region is always greater than the sphere radius of the outer face of the socket insert; and that the socket insert be self-lockingly clampable in the receiving space. By virtue of the above-mentioned linear contact between socket insert and socket shell, the socket insert can readily be rotated and tilted in the receiving space of the socket shell in order to align the socket insert in its position in the optimum way. As soon as the socket insert has been aligned, light pressure is sufficient to press the socket insert into the tapering receiving space, whereupon the socket insert is clamped self-lockingly in the receiving space. The self-locking clamping effects fixation of the socket insert in the socket shell with a high degree of stability. A load acting on the joint brings about an additional pressing of the socket insert into the socket shell, so that the fixation of the socket shell is additionally strengthened. Because the optimally aligned socket insert is fixated by simply being pushed into the receiving space, that fixation is simple to carry out and does not require any additional instruments or additional fixing means. The self-locking clamping comes into effect after a minimum displacement path of the socket insert into the receiving space, so that when the socket insert is being fixated unintentional displacement of the alignment of the socket insert cannot occur. As regards further advantages of the above-mentioned construction, reference is made to DE 103 60 390 A1. That applies, in particular, to the advantage that in the event of impingement of the femoral neck of a prosthesis stem on the edge of the joint socket, even in the worst case only the socket insert is loosened in the socket shell and not the socket shell in the bone. When the joint is subsequently subjected to a normal load, the socket insert is then pressed into the receiving space of the socket shell again and is clamped and fixated afresh.

Proceeding on the basis of the last-mentioned prior art and retaining the advantages thereof, the present invention is now based on the problem of providing a joint socket, especially a joint socket for a hip endoprosthesis, which achieves an increased clamping action between socket shell and socket insert, with care being taken at the same time that the specific pressure between the mutually corresponding clamping faces is reduced.

That problem is solved according to the invention by the characterizing features of claim 1. It is thereby possible to increase the contact area of the mutually corresponding clamping faces and to reduce the specific pressure in the region of the mutually corresponding clamping faces, without the clamping action being impaired as a result.

An important aspect of the present invention is therefore that the resistance to deformation of at least one of the mutually corresponding faces of the receiving space and the socket insert is reduced with respect to that of the core region of those components, especially as a result of the formation of a surface structure. As a consequence, increased deformability of at least one of the mutually corresponding clamping faces is obtained, with the result that the contact area is increased and the specific pressure is correspondingly reduced, without there being any adverse effect on the clamping action.

In a preferred embodiment, the surface structure according to the invention is obtained as a result of the surface in question's having ribs or like protuberances extending around the periphery of the receiving space and/or the socket insert. Preferably, the ribs are triangular, i.e. pitched-roof-like, in cross-section.

In accordance with the prior art according to DE 103 60 390 A1, the inner face of the receiving space can be conical in the region of the line of contact, that is to say can have an infinite radius of curvature.

The angle of taper of the inner face is about from 3.5° to 12°, especially about from 5° to 8°, that being a preferred range for a so-called “self-locking angle” which depends upon the material pairing. That angle is the angle between the cone centre axis and the cone generator.

By virtue of the surface structure of at least one of the mutually corresponding faces it is possible for the socket insert and the socket shell each to be manufactured from a hard material. In that case too, the surface structure gives rise to the lower resistance to deformation of that face required according to the invention. The socket shell is preferably made of a biocompatible metal, for example a titanium alloy. For the socket insert it is possible to choose a material in accordance with the tribological pairing of socket shell and joint head, for example a metallic or ceramic material or alternatively plastics material.

Depending upon the metal pairings it can be advantageous for the peripheral ribs or peripheral protuberances in each case to have breaks distributed uniformly around the periphery.

In an especially advantageous embodiment, the surface structure of the at least one of the mutually corresponding faces consists of grooves of triangular cross-section which are separated by ribs running in the peripheral direction, which ribs likewise have a triangular or also possibly trapezoidal cross-section.

A preferred embodiment of a joint socket constructed in accordance with the invention, or rather a portion thereof, is described in detail below with reference to the accompanying drawings.

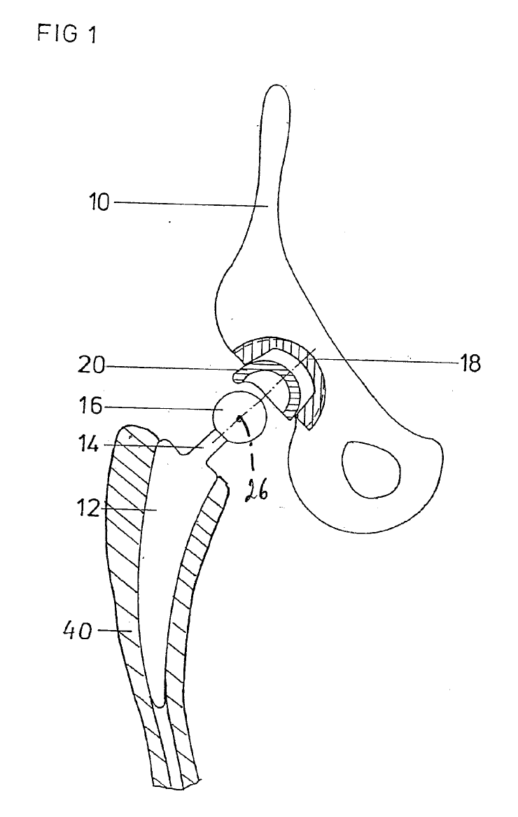

FIG. 1 shows a hip total endoprosthesis, partly in axial section, partly in side view;

FIG. 2 shows a portion of the joint socket of the prosthesis according to FIG. 1 in axial section;

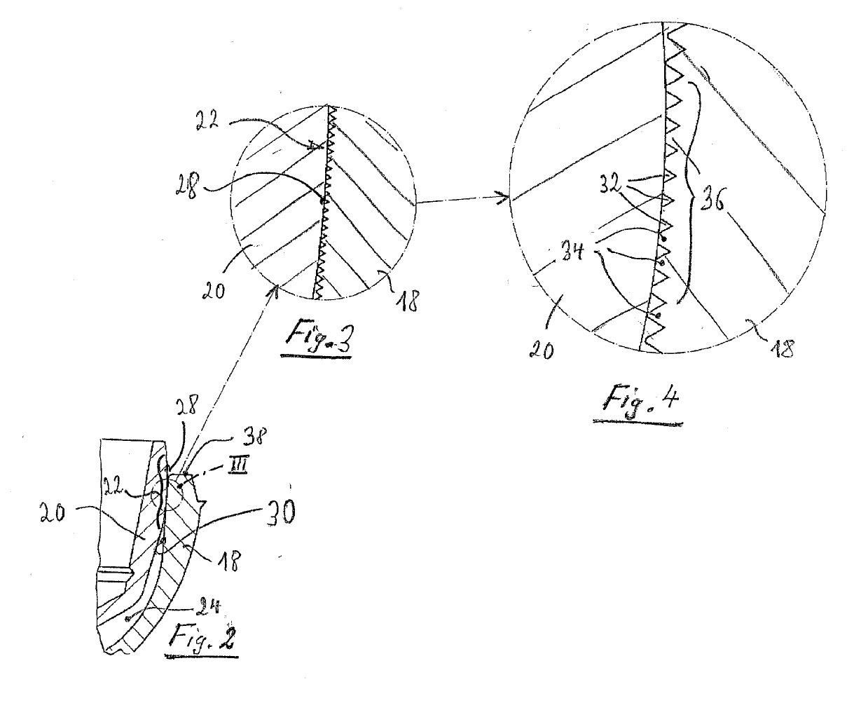

FIG. 3 shows an enlarged detail III of the joint socket according to FIG. 2 on an enlarged scale; and

FIG. 4 shows the detail in accordance with FIG. 3 on a further enlarged scale.

The hip total endoprosthesis shown diagrammatically in FIG. 1 consists of a joint socket implantable in a pelvic bone 10 and a prosthesis stem 12 which is installed in the femur 40. The prosthesis stem 12 has a femoral neck 14 on which there is arranged a joint head 16 which is mounted in the joint socket. The joint socket consists of a socket shell 18, which is positionable in the pelvic bone 10, and a socket insert 20. The socket shell 18 can be fixated in the pelvic bone 10 by means of additional screws. The socket shell 18 can also be in the form of a screw-in socket having a thread on its outer face. The substantially hemispherical socket shell 18 defines a receiving space 24 which is rotationally symmetrical with respect to the centre axis 26 of the socket shell 18.

The socket insert 20 has a portion 22 having a spherical outer face 28 which corresponds to a conical or likewise spherical inner face 30 of the receiving space 24 in such a way that the two above-mentioned faces are in contact concentrically with the rotational axis 26. In the region of that concentric contact, the radius of curvature of the inner face 30 of the receiving space 24 of the socket shell 18 is always greater than the radius of curvature of the spherical portion 22 of the socket insert 20, so that the latter is held self-lockingly clampable in the receiving space 24 of the socket shell 18. When the inner face 30 is a peripheral face that tapers conically towards the pole of the socket shell 18, the radius of curvature is infinite.

It is of considerable importance that the resistance to deformation of at least one of the mutually corresponding faces 28, 30 of socket insert 20 and receiving space 24 is reduced with respect to that of the core region of those components, especially as a result of the formation of a surface structure. In the embodiment shown in FIGS. 2 to 4, such a structure is provided on the spherical inner face 30 of the receiving space 24, more specifically in the form of grooves 32 of triangular cross-section extending around the periphery, which grooves are separated by ribs 34 likewise of triangular cross-section running in the peripheral direction. In the region 36 of greatest pressure between socket insert 20 and socket shell 18, the tips of the peripheral ribs 34 become deformed, as can be seen very clearly in FIG. 4. Those tips are therefore responsible for the lower resistance to deformation of the inner clamping face 30 of the receiving space 24 of the socket shell 18.

As a result of the mentioned deformation, the ribs 34 acquire a more or less marked trapezoidal cross-section.

Unlike the prior art according to DE 103 60 390 A1, the faces 28, 30 are in contact not along a line of contact concentric with the rotational axis 26 of the receiving space 24 but along a contact strip concentric with the rotational axis 26 of the receiving space 24. As a result, the specific pressure between the two above-mentioned faces can be considerably reduced, without the clamping action being lost. On the contrary, it has been found that the described measures even achieve a greater clamping action, without any risk of unacceptable deformation of the basic body of socket shell and/or socket insert.

It should additionally be pointed out that the angle enclosed by the inner face 30 adjoining the equatorial plane 38 of the socket shell and the rotational axis 26 is chosen, depending upon the material pairing of socket shell 18 and socket insert 20, so that self-locking is achieved, that self-locking being additionally increased by the afore-mentioned surface structure. Preferably, that angle is about from 3.5° to 12°, especially about from 5° to 8°. In the case of a metallic socket shell 18, for example, a self-locking angle of taper for a metallic socket insert 20 is about from 4.0° to 5.0° and a self-locking angle of taper for a ceramic socket insert 20 is about from 8.0° to 10.0°.

It should also be pointed out that it can be advantageous for the face of the two mutually corresponding contact or clamping faces not having a prominent surface structure to be at least surface-roughened with an average roughening value Rₐ (according to DIN 4760 or VSM 10321) of from 0.5 to 3.5μ, thereby achieving even better meshing with the structured surface.

All the features disclosed in the application documents are claimed as being important to the invention, provided that they are novel over the prior art, either individually or in combination.

### REFERENCE NUMERALS

10 pelvic bone
12 prosthesis stem
14 femoral neck
16 joint head
18 socket shell
20 socket insert
22 spherical portion of the socket insert
24 receiving space
26 centre axis (rotational axis) of the socket shell
28 spherical outer face of the socket insert
30 conical or spherical inner face of the receiving space 24
32 peripheral groove
34 peripheral rib
36 clamping region of the socket insert inside the socket shell
38 equatorial plane of the socket shell

1-8. (canceled)

9. A joint socket for a hip joint endoprosthesis, comprising:
a socket shell configured for implantation in a bone and comprising a receiving space comprising an inner face; and
a socket insert coupleable to the socket shell and configured to accommodate a joint head of a hip prosthesis stem therein, the socket insert comprising a spherical portion comprising a spherical outer face, the spherical portion configured to be received by the receiving space so that the spherical outer face and inner face contact each other concentrically with a rotational axis of the socket shell, the radius of curvature of the inner face being greater than the radius of curvature of the spherical portion in a region of concentric contact, such that the spherical portion self-lockingly clamps in the receiving space,
wherein a resistance to deformation of one or more of the inner face and outer spherical face is less than that of a core region of the corresponding socket shell or socket insert.

10. The joint socket of claim 9, wherein a surface structure on one or more of the inner face and spherical outer face reduces the resistance to deformation of at least one of the faces.

11. The joint socket of claim 10, wherein the surface structure is configured to reduce pressure between the contact surfaces.

12. The joint socket of claim 9, wherein a surface structure of one or more of the inner face and outer face comprises ribs or protuberances extending around a periphery of the receiving space and/or the socket insert.

13. The joint socket of claim 12, wherein the ribs or protuberances are triangular in cross-section.

14. The joint socket of claim 9, wherein the inner face is conical in the region of concentric contact between the socket shell and socket insert.

15. The joint socket of claim 9, wherein an angle the inner face makes relative to the rotational axis is a self-locking angle for the material pairing of the socket shell and the socket insert.

16. The joint socket of claim 15, wherein the angle of the inner face is between 3.5° and 12° relative to the rotational axis.

17. The joint socket of claim 15, wherein the angle of the inner face is between 5° and 8° relative to the rotational axis.

18. The joint socket of claim 12, wherein the ribs or protuberances comprise breaks distributed uniformly about the rotational axis.

19. The joint socket of claim 9, wherein the surface structure of one or more of the inner and outer faces comprises grooves of triangular cross-section separated by ribs that extend about the rotational axis.

20. The joint socket of claim 19, wherein the ribs comprise a triangular cross-section.

21. The joint socket of claim 19, wherein the ribs comprise a trapezoidal cross-section.

22. A hip joint endoprosthesis, comprising:
a prosthesis stem configured for implantation in a femur and having a joint head;
a socket shell configured for implantation in a pelvic bone and comprising a receiving space comprising an inner face; and
a socket insert coupleable to the socket shell and configured to accommodate the joint head therein, the socket insert comprising a spherical portion comprising an outer face, the spherical portion configured to be received by the receiving space so that the outer face and inner face contact each other concentrically with a rotational axis of the socket shell, the radius of curvature of the inner face being greater than the radius of curvature of the spherical portion in a region of concentric contact, such that the spherical portion can self-lockingly clamp in the receiving space,
wherein the resistance to deformation of one or more of the inner face and outer face is less than that of a core region of the corresponding socket shell or socket insert, a surface structure on one or more of the inner face and spherical outer face configured to reduce the resistance to deformation of one or more of the faces in the socket shell and socket insert.

23. The endoprosthesis of claim 22, wherein the surface structure of one or more of the inner face and outer face comprises ribs or protuberances extending around a periphery of the receiving space and/or the socket insert.

24. The endoprosthesis of claim 23, wherein the ribs or protuberances are triangular in cross-section.

25. The endoprosthesis of claim 22, wherein the inner face is conical in the region of concentric contact.

26. The endoprosthesis of claim 23, wherein the ribs or like protuberances have breaks distributed uniformly around the periphery.

27. The endoprosthesis of claim 22, wherein the surface structure of one or more of the inner and outer faces comprises grooves of triangular cross-section separated by ribs running about the rotational axis.

28. The endoprosthesis of claim 27, wherein the ribs comprise a triangular cross-section.

29. A method for implanting a hip joint endoprosthesis, comprising:
inserting a socket shell in a pelvic bone, the socket shell having an inner surface that defines an accommodating space extending about an axis of rotation, wherein a surface structure on the inner face reduces the resistance to deformation of a portion of the inner surface relative to a core region of the socket shell;
inserting a socket insert into the accommodating space so that an outer surface of the socket insert contacts the conical inner surface; and
pressing the socket insert into the accommodating space to engage the socket insert with the socket shell in a self-locking manner such that the surface structure results in a reduction in pressure between the inner and outer surfaces.

30. The method of claim 29, further comprising the steps of:
inserting a prosthesis shaft into a femur corresponding to the pelvic bone; and
aligning the socket shell and a head of the prosthesis shaft.

31. The method of claim 29, wherein the inner face is conical in the region of concentric contact.

32. The method of claim 29, wherein an angle the inner face makes relative to the axis of rotation is the self-locking angle for the material pairing of the socket shell and the socket insert.

33. The method of claim 32, wherein the angle of the inner face is between 3.5° and 12° relative to the rotational axis.

34. The method of claim 32, wherein the angle of the inner face is between 5° and 8° relative to the rotational axis.

35. The method of claim 29, wherein:
the surface structure comprises peripheral ribs; and
the step of inserting comprises deforming the peripheral ribs.

36. The method of claim 35, wherein the peripheral ribs comprise a triangular shape and the deforming of the peripheral ribs comprises deforming the triangular shape to a trapezoidal shape.
